(19) United States
(12) Patent Application Publication
(10) Pub. No.: US 2018/0166176 A1
(43) Pub. Date: Jun. 14, 2018

(54) SYSTEMS AND METHODS OF AUTOMATED ACCESS INTO A TELEHEALTH NETWORK

(71) Applicant: Wellspring Telehealth, LLC, Bryan, TX (US)

(72) Inventors: Menville B. Flippen, College Station, TX (US); Steven F. Gaffney, Westlake, TX (US); Robert Peyton Reaves, Fort Worth, TX (US)

(73) Assignee: Wellspring Telehealth, LLC, Bryan, TX (US)

(21) Appl. No.: 15/580,930

(22) PCT Filed: Jun. 9, 2016
(86) PCT No.: PCT/US16/36549

§ 371 Date: Dec. 8, 2017

Related U.S. Application Data
(63) Continuation-in-part of application No. 15/176,900, filed on Jun. 8, 2016.
(60) Provisional application No. 62/174,893, filed on Jun. 12, 2015.
(60) Provisional application No. 62/315,200, filed on Mar. 30, 2016.

(51) Int. Cl. G16H 80/00; G16H 40/20; G16H 10/60; A61B 5/00; A61B 5/01; A61B 5/024

(52) U.S. Cl. CPC G16H 80/00; G16H 40/20; A61B 5/024; A61B 5/0022; A61B 5/01; G16H 10/60

(57) ABSTRACT

A system and method for automatically controlling and providing access to a telehealth network, wherein the automatic access is triggered based upon at least one event. A trigger enables access to the telehealth network. A second trigger disables access to the telehealth network. Also, a telehealth system wherein access is automatically activated by activation of a SIM card or chip within a mobile communications device.

### CROSS REFERENCES TO RELATED APPLICATIONS

This application claims the benefit of U.S. Provisional Application No. 62/174,893, filed Jun. 12, 2015, U.S. Provisional Application No. 62/315,200, filed Mar. 30, 2016, and U.S. Non-Provisional application Ser. No. 15/176,900, filed Jun. 8, 2016, each of which is incorporated herein by reference in their entirety.

### BACKGROUND OF THE INVENTION

### 1. Field of the Invention

The present invention is generally directed to automated access to a telehealth network, and more particularly to providing automated access to a telehealth network based upon telecommunications SIM card or chip activation within a mobile communications device and integrative medical, wellness, and behavioral analysis within the telehealth network.

### 2. Description of the Prior Art

Generally, telemedicine provides for the remote diagnosis and treatment of patients by means of telecommunications technology. This technology has been realized for primary care and specialist services, remote patient monitoring, consumer medical and health information, and medical education. Mechanisms for these services include devices capable of capturing diagnostic images and vital signs, such as blood glucose and electrocardiograms, and transmitting them to a physician provider or central data storage; this classic example of telemedicine is shown in PRIOR ART FIG. 1. Together, this technology can reach numerous outlying clinics, community centers, and individual patients, and provides improved healthcare access, reduced cost, and better quality. A major benefit to telehealth technology is the reduction in travel to and wait-times at clinics. Further, this convenience in healthcare may extend to patients who are traveling so they may connect instantly with a network of physicians for information, advice, and treatment, including prescription medication, when appropriate.

More recently, efforts have been made to provide more than pharmaceutical-based prescription treatment using telehealth networks. Telehealth networks can accept and communicate patient-specific data, such as health history, diet, exercise, medicine, and stress. With a whole-person perspective, telehealth technology can now monitor a patient, analyze his/her data, and consult or guide the patient on complete wellness. This style of complete and individualized healthcare is increasingly demanded.

Also, it is known in the art to provide network-based inquiry for medical information that is generic, i.e., not specific to an individual through medical evaluation, diagnosis, etc. and not provided by licensed healthcare professionals. Individuals may use web-based medical information for self-diagnosis and/or treatment. However, reliability and quality of information, and risk of misdiagnosis or mistreatment are issues with patient-based internet medical information research.

Representative prior art references include the following:

U.S. Pat. No. 5,832,488 for Computer system and method for storing medical histories using a smartcard to store data by inventor Eberhardt filed Jan. 22, 1997 and issued Nov. 3, 1998 describes a computer system and method for programming it for storage of individual medical histories on a storage device, preferably about the size of a credit card, for adding new medical data about the individual to the device and for communicating with other computers to retrieve large data records about the individual; and for enabling a second computer to collate and sort data relating to selected medical fields from the data of such individual and from the data about other individuals transferred to the second computer.

U.S. Pat. No. 7,613,481 for Method for the management of information stored on a smart card readable by a mobile telephone by inventor Aubert filed Sep. 13, 2004 and issued Nov. 3, 2009 describes a method of managing information from a telecommunication equipment unit comprising a screen and equipped with a SIM memory card connector comprising recording the information in a memory register of a standard SIM card, and exchanging information between the telecommunication equipment unit and the SIM card according to a standardized protocol, without subscriber authentication and connection to a telecommunication network, and wherein a SIM Toolkit application developed in the SIM card is executed, thereby enabling management of the information.

U.S. Pat. No. 7,593,952 for Enhanced medical treatment system by inventors Soll, et al. filed Apr. 9, 1999 and issued Sep. 22, 2009 describes an enhanced medical treatment system which seeks input from the patient and the physician about the medical problems faced by the patient. It analyzes this information and seeks to guide physicians to a correct diagnosis of the complaint. It also seeks to educate the patient about his/her medical problems and provides information about the problem. It is able to store all this information, so that a continuous record of the patient's visits and problems is kept on file and the physician is able to utilize the medical history to solve the patient's current problems. With this system in place the medical establishment will be able to improve health care delivery to the patients and be able to better manage the process of the providing health care. Furthermore, this system will also lower the cost of providing health care without compromising on the quality of health care.

U.S. Pat. No. 7,310,734 for method and system for securing a computer network and personal identification device by inventors Boate and Reed filed Feb. 1, 2001 and issued Dec. 18, 2007 discusses an improved computer network security system and a personal identifier device used for controlling network with real time authentication of both a person's identity and presence at a computer workstation is provided. A new user is registered to a portable personal digital identifier device and, within the portable personal digital identifier device, an input biometric of the user is received and a master template is derived therefrom and securely maintained in storage. A private key and public key encryption system is utilized to authenticate a user registered to the portable personal digital identifier device. The personal digital identifier device verifies the origin of a digitally signed challenge message from the network security manager component. A digitally and biometrically signed challenge response message is produced and transmitted by the personal digital identifier device in response to the verified challenge message.

U.S. Pat. Nos. 7,315,825 and 7,395,216 for using predictive models to continuously update a treatment plan for a patient in a healthcare location by inventors Rosenfeld and Breslow are directed to a system for determining a treatment plan for a patient comprises a database of patient data elements indicative of a medical condition associated with a patient. A predictive model is applied to patient assessment data and used to prepare a treatment plan. A rules engine applies a patient rule consistent with the treatment plan to selected data elements stored in the database to produce an output indicative of a change in the medical condition of the patient. The output from the rules engine is used to determine if intervention is warranted. The predictive model is applied continuously to determine whether to update the treatment plan and, if necessary, the patient rule.

U.S. Pat. Nos. 7,624,028 and 7,853,455 for remote health monitoring and maintenance system by inventor Brown are directed to a system and method that enables a health care provider to monitor and manage a health condition of a patient. The system includes a health care provider apparatus operated by a health care provider and a remotely programmable patient apparatus that is operated by a patient. The health care provider develops a script program using the health care provider apparatus and then sends the script program to a remotely programmable patient apparatus through a communication network such as the World Wide Web. The script program is a computer-executable patient protocol that provides information to the patient about the patient's health condition and that interactively monitors the patient health condition by asking the patient questions and by receiving answers to those questions.

U.S. Pat. No. 7,684,999 for user-based health monitoring by inventor Brown filed Dec. 3, 2004 and issued Mar. 23, 2010 discusses a user-based monitoring system that includes a remote user-based subsystem with at least one display and at least two microprocessor-based units in communication with each other. The subsystem is configured to facilitate collection of user-related data. The system also has at least one central server remotely located from and configured for two-way communication with the user-based subsystem so that it can receive and deliver signal communications to and from the user-based subsystem. The system is suited, amongst others, for monitoring remotely the health of a system user.

U.S. Pat. No. 7,860,725 for Method for remote medical consultation and care by inventors Gopinathan, et al., filed Dec. 5, 2002 and issued Dec. 28, 2010 discusses a method for remote medical consulting includes collecting diagnostic data using at least one wearable device contoured to at least a portion of a person's hand, transmitting the diagnostic data to a remote location, transmitting audio data and video images of the patient to the remote location, and communicating diagnosis and/or treatment information to the patient based at least in part on the diagnostic data. The treatment information may include a prescription electronically transmitted to the patient or a pharmacy. The method includes billing of the patient via credit or debit card, bank account, or a third party, such as an insurance company. The diagnostic data as well as the audio and video data may be transmitted wirelessly via cellular or satellite communication networks and/or using a wide area computer network such as the internet.

U.S. Pat. No. 8,762,173 for Method and apparatus for indirect medical consultation by inventors Wasson, et al., filed Nov. 13, 2008 and issued Jun. 14, 2014 discusses a method and apparatus is provided comprising a technology engine designed to facilitate indirect medical consultation with health care providers and create a medical record of this indirect consultation event. In one embodiment is provided a method and apparatus for capturing electronically one or more of the following: (a) patient complaints; (b) health care provider's impression/review; (c) health care provider/patient discussion of complaint symptoms following which a treatment plan is formulated regarding health care strategy; (d) any prescription issued by the health care provider; and/or (e) a set of follow-up visits by the patient.

U.S. Pat. No. 8,941,659 for Medical symptoms tracking apparatus, methods and systems by inventors Dawson, et al., filed Jan. 30, 2012 and issued Jan. 27, 2015 discusses in an aspect, a method of monitoring one or more symptoms of a person include repeating, over a period of time, the steps of: selecting, by the person, one or more symbolic representations corresponding to one or more symptoms from a predefined set of symbolic representations presented to the person; and electronically recording data regarding the one or more symbolic representations selected by the person such that the data is electronically accessible later for generating a history of the symptoms of the person over the period of time. The one or more symbolic representations corresponding to one or more symptoms is selected using an electronic device having a component for displaying the predefined set of symbolic representations that is coupled to a user input for receiving the selection of the one or more symbolic representations by the person.

US Patent Application Publication No. 2007/0255115 for Remote diagnostic & treatment system by inventors Anglin, et al., filed Apr. 27, 2006 and published Nov. 1, 2007 discusses Methods and apparatus for providing remote diagnosis and treatment are disclosed. One embodiment of the invention comprises a cellular telephone (16A) that includes a camera (34), a display (40), a speaker (36A), a microphone (36B) and embedded diagnostic and treatment software (18). An alternative embodiment may also include one or more data devices (88) that may be connected to the cellular phone (16A) using a wireless (30A) or wired (30B) connection.

US Patent Application Publication No. 2007/0088580 for Systems and methods for providing comparative health care information via a network by inventor Richards filed Aug. 15, 2006 and published Apr. 19, 2007 discusses methods and systems for providing comparative health care information via a network. One embodiment of the invention can comprise a comparative information application engine. The comparative information application engine can be adapted to receive claim-related information. Claim-related information can be stored in a database or other data storage device. Cost information can be determined from at least a portion of the claim-related information. Provider information can be determined from at least a portion of the claim-related information. In this embodiment, the claim-related information can be processed by an automated disclosure processing application program. In response to the request for statistical information, an output can be generated based at least in part on the provider information or the cost information.

US Patent Application Publication No. U.S. Pat. No. 7,818,183 for connecting consumers with service providers by inventor Schoenberg filed Oct. 22, 2008 and issued Oct. 19, 2010 is directed to a computerized system that provides information and services to the consumers in addition to connecting them with providers. The computerized system includes an access control facility, which manages and controls whether a given consumer may access the system and what level or scope of access to the features, functions, and services the system will provide.

U.S. Pat. No. 7,827,040 for patient-control of health related data in a remote patient monitoring system by inventor Brown filed Sep. 16, 2003 and issued Nov. 2, 2010 is directed to a networked health-monitoring system for collecting and processing patient health-related data. A plurality of remote patient sites each includes at least one display, a data management unit configured to facilitate collection of patient health-related data, a memory, and stored program instructions for generating health-monitoring related information on the display.

U.S. Pat. No. 7,953,613 for a health maintenance system by inventor Gizewski filed Jan. 3, 2007 and issued May 31, 2011 discusses a health maintenance system for comprehensive health assessment, abnormality detection, health monitoring, health pattern and trend detection, health strategy development, and health history archiving. The health maintenance system comprises a subscriber segment and a system segment, communicatively coupled.

U.S. Pat. No. 7,970,620 for multi-user remote health monitoring system with biometrics support by inventor Brown filed Aug. 30, 2002 and issued Jun. 28, 2011 is directed to a networked system for identifying an individual, communicating information to the individual, and remotely monitoring the individual. The system includes a remotely programmable apparatus that occasionally connects to a server via a communication network such as the Internet. The remotely programmable apparatus interacts with the individual in accordance with a script program received from the server. Among other capabilities, the script program may instruct the remotely programmable apparatus to identify the individual, to communicate information to the individual, to communicate queries to the individual, to receive responses to the queries, and to transmit information identifying the individual and the responses from the remotely programmable apparatus to the server.

U.S. Pat. No. 7,970,633 for system and method for delivering medical examination, treatment and assistance over a network by inventor Bulat filed Nov. 18, 2008 and issued Jun. 28, 2011 is directed to a system for delivering medical examination, diagnosis, and treatment services from a health care practitioner to a patient over a network includes a plurality of health care practitioner terminals and a plurality of patient terminals in audiovisual communication over the network with any of the plurality of health care practitioner terminals. Each of the plurality of health care practitioner terminals includes a display device. The system also includes a call center in communication with the patient terminals and the health care practitioner terminals. The call center routs a call from a patient at one of the patient terminals to an available health care practitioner at one of the health care practitioner terminals so that the available health care practitioner may carry on a two-way conversation with the patient and visually observe the patient. The available health care practitioner may make an assessment of the patient and treat the patient.

U.S. Pat. No. 8,249,894 for networked remote patient monitoring with handheld devices by inventor Brown filed Sep. 16, 2003 and issued Aug. 21, 2012 is directed to a networked health-monitoring system is configured to collect and process patient health-related data. The system includes at least one handheld microprocessor device having a display; and a memory. At least one central server is connected to receive health-related data communicated to the handheld microprocessor device and at least one remotely located health care professional computer is in signal communication with the central server. It receives patient health-related information based on the patient health-related data received from the handheld microprocessor device. The system is configured to convert the handheld microprocessor device into a healthcare monitoring device that functions to monitor patient health conditions and communicate data related to the monitored conditions to the central server.

U.S. Pat. No. 8,788,715 for network and interface selection on a computing device capable of establishing connections via multiple network communications media by inventors Krantz, et al. filed Jul. 22, 2011 and issued Jul. 22, 2014 is directed to a system and method for carrying out network and interface selections across multiple media is disclosed, wherein the disclosed system facilitates automated network interface configuration decision-making that spans a set of networks supporting communications via differing media.

US patent application 2010/094659 for consolidation of consumer interactions within a medical brokerage system by inventor Schoenberg filed Dec. 15, 2009 and published Apr. 15, 2010 discusses a health improvement function to assess a consumer patient's current overall health and wellness state, a specific area of the health and wellness state, or treatment for a specific condition, issue or symptom. A profiling operation is performed using the data collected by the intake stage to form a profile of the patient. These data include the consumer's goals, where the consumer wants the consumer's health state to be in the future, and desired changes in the consumer's overall health and wellness state or in a specific area of the consumer's health and wellness state (e.g., body weight, BMI, cholesterol level, etc.), or treatment for a specific condition, issue or symptom.

Regarding telehealth technology, the prior art is directed to incentives that reward favorable health, interfacing with external data sources to acquire information from available systems automatically to use the information to prepare providers at the beginning of an engagement, consumer profiles and analytics of consumer data, and secured access into a network or management of data files in a network.

The prior art is limited regarding patient actions automatically generated by forecasting algorithms based on a patient's patterns, integrating a patient's information with the patient's profile to generate patient patterns, and analysis that is automated. Further, automated, intermittent, controlled access to a telehealth network has not been realized. The development of such a telehealth network will allow for a more complete and convenient healthcare.

### SUMMARY OF THE INVENTION

The present invention is directed to systems and methods for providing remote access into and out of a telehealth network, wherein the remote access is automatic and based upon at least one trigger event. In one embodiment, the trigger event includes activation of a telecommunications SIM card or chip in a mobile phone, a smartphone, or a computer device having network-based access for interactive communication with at least one remote server computer for providing telehealth information services to the user following activation of the SIM card or chip for the duration of the life of the SIM card or chip, e.g., a predetermined period of time or a predetermined monetary equivalent of network access for a prepaid SIM card or chip, wherein the activation of the SIM card or chip by insertion within a mobile phone, smartphone or computer device as described herein, provides a trigger for activation of and access to the telehealth services.

These and other aspects of the present invention will become apparent to those skilled in the art after a reading of the following description of the preferred embodiment when considered with the drawings, as they support the claimed invention.

### BRIEF DESCRIPTION OF THE DRAWINGS

PRIOR ART FIG. 1. A schematic diagram of a typical telehealth network communication between a patient device and network.

FIG. 2. A schematic diagram of communication lines for a telehealth network of the present invention.

FIG. 3. A flow diagram illustrating steps in a short term eligibility process according to the present invention.

FIG. 4. A schematic diagram illustrating a general cloud-based computer system.

### DETAILED DESCRIPTION

The present invention provides a system for automated, trigger-based activation for and access to a telehealth network including medical information provided within a closed, member-based or membership-based system, wherein the membership or member activation is associated with activation of a SIM card or chip for a mobile communications device operable for network-based or telecommunications-based access to at least one remote server having medical information about symptoms and associated medical conditions, diseases, and/or illnesses and corresponding treatment information.

The present invention is directed to systems and methods for providing remote access into and out of a telehealth network, wherein the remote access is automatic and based upon at least one trigger event. In one embodiment, the trigger event includes activation of a telecommunications SIM card or chip in a mobile phone, a smartphone, or a computer device having network-based access for interactive communication with at least one remote server computer for providing telehealth information services to the user following activation of the SIM card or chip for the duration of the life of the SIM card or chip, e.g., a predetermined period of time or a predetermined monetary equivalent of network access for a prepaid SIM card or chip, wherein the activation of the SIM card or chip by insertion within a mobile phone, smartphone or computer device as described herein, provides a trigger for activation of and access to the telehealth services.

SIM card or chip technology is described in U.S. Pat. No. 7,613,481 for Method for the management of information stored on a smart card readable by a mobile telephone by inventor Aubert, which is incorporated herein by reference in its entirety. By way of contrast to this prior art reference, the present invention provides for automatic access to the telehealth network via the telecommunications network upon activation of the SIM card or chip within the mobile communications device.

Additional technology for telehealth network systems and methods may include the following, each of which is incorporated herein by reference in its entirety:
1) Smart Card/SIM Technology is described in a whitepaper entitled “The SIM: The Key to Better Healthcare?” published by GSMA in 2012 available via the following web link: http://www.gsma.com/connectedliving/wp-content/uploads/2012/03/uicc20whitepaper.pdf. It describes SIM card or chip technology. More than five billion devices around the world are authenticated on mobile networks using an embedded SIM (an application running on a Universal Integrated Circuit Card or UICC), which stores and presents a unique identifier for each customer to their mobile operator. SIM-style solutions could provide increased security for, and access to, healthcare providers' information and communications technology (ICT) systems, which can readily harness the strong identity management capabilities of a UICC. In most countries, healthcare ICT systems lack a widely-deployed identity management technology, so there is a clear opportunity to satisfy a critical market need.
2) SIM cards for cell phones are a subset of smart cards. Smart cards are widely adopted for authentication in credit cards and the technology is available for US healthcare http://www.smartcardalliance.org/resources/pdf/SmartCard_Technology_in_Healthc are_FAQ_FINAL_096012.pdf
3) Electronic Health Care Cards described in the following http://www.gi-de.com/gd_media/media/en/documents/brochures/mobile_security_2/gesundheitskart e/Smart-Cards-for-Future-Healthcare-Systems.pdf and
4) https://www.technologyreview.com/s/424281/medical-smart-cards-find-their-niche/
5) Cell phone technologies with SIM and medical including GPS and SIM https://www.medi-alert.com/gps-program-2
6) Smart watch phones with SOS−SIM+GPS+heart rate monitoring http://jedy.en.alibaba.com/product/60319510335-801374674/2015_best_selling_products_forelderly_heart_rate_GPSmonitoring_SO S_health_smart_watch_with_sim_card_watch_phone.html
7) HIPAA and mobile device communication http://www.americanbar.org/newsletter/publications/abahealthesourcehome/abah ealth_lawe_source_1110_barrett.html

In another embodiment, systems and methods provide for automatic, and intermittent or occurring occasionally or at regular or irregular intervals or periodic access, to a telehealth network, wherein the telehealth network provides automated analysis of inputs from a remote user via a mobile device having a pre-authorized, pre-enrolled, or pre-authenticated access to the telehealth network having at least one server computer and database of medical information, wherein the inputs include a patient's health history, including diet, exercise, and behavior, user-identified symptoms, observations, descriptions, and/or data and wherein the automated analysis of the inputs includes matching with predetermined medical conditions, illnesses, injuries, diseases, and combinations thereof for providing non-diagnostic outputs for users to make decisions or take actions to seek professional medical care or not, based upon risks, location, access to medical care, access to medical facilities, treatments, equipment, and combinations thereof.

By way of example, in one embodiment of the present invention, a traveler acquires a pre-authorized and/or prepaid SIM card or chip for a mobile communications device, e.g., a mobile phone, activates the SIM card or chip by inserting it into the mobile communications device and powering on the device, which automatically triggers enrollment and/or activation into the telehealth network associated with the SIM card or chip and/or the telecommunications service provider associated therewith. The membership or authorized access to the telehealth network is active following the trigger event and for the duration of the SIM card or chip activation, e.g., predetermined period of time or prepaid amount of time of access to the telecommunications network via the mobile communications device with the SIM card or chip inserted therein.

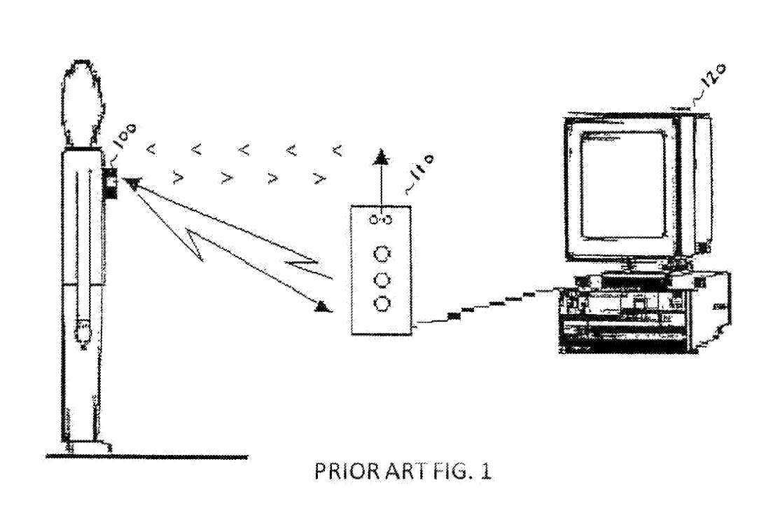

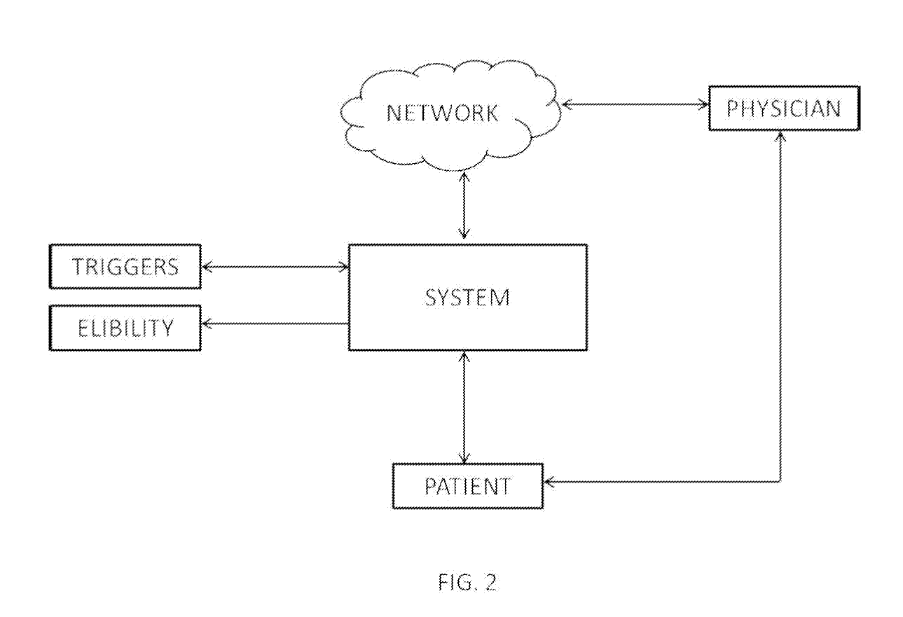

In a preferred embodiment of the present invention, the system and methods provide automated, controlled access to a telehealth network to a user of a mobile communications device with a prepaid or pre-authorized SIM card or chip, which when activated within the mobile communications device provides for provides access to the telehealth network. FIG. 2 illustrates a system configuration for an embodiment of the present invention wherein the mobile communications device is illustrated in network-based communication with at least one remote server computer and database having medical information stored thereon. A user or patient directly requests access to the system that controls entry into the telehealth network via the mobile communications device with the SIM card or chip activated and operatively connected within the device. The system automatically confirms the user eligibility based upon the SIM card or chip and thereby provides for the user to have remote access into the telehealth network for providing medical information inputs that are matched to corresponding medical information including medical conditions, illnesses, injuries, diseases, and combinations thereof for providing non-diagnostic outputs for users to make decisions or take actions to seek professional medical care or not, based upon risks, location, access to medical care, access to medical facilities, treatments, equipment, and combinations thereof.

User inputs are provided via the mobile communications device having the prepaid or pre-authorized SIM card or chip activated therein. The inputs are provided with automatic identification associated with the SIM card or chip, such as a unique identification number or account associated with the SIM card or chip, via a website, mobile application (App), phone call, text message, SMS, or telecommunications connection.

In an alternative embodiment, access is provided with fulfillment of pre-requisites to be admitted into the telehealth network. Approval into the system (i.e., pre-requisite fulfillment and/or registration) requires inputs of patient identification and information, such as, by way of example and not limitation, a unique identification number, ticket purchase, or group association. System eligibility confirmation is followed by patient verification, wherein the patient inputs more specific identifiable information, such as a unique identification number, via a website, mobile application (App), phone call, text message, SMS, or telecommunications connection.

Preferably, verification is made through the mobile communications device with the SIM card or chip and further includes a location device and/or data transceiver for geolocation of the user at the time of inputs transmitted by the mobile communications device via the telecommunications network or other network-based communication to the at least one remote server computer and database for the telehealth network. This verification confers to the system automatic real-time or near-real-time matching of the inputs with medical information stored on the system for providing outputs communicated to the mobile communications device for informational briefs that are non-diagnostic, i.e., not medical diagnosis by a medical practitioner or medical advice. In another embodiment wherein the identification of the user is linked automatically and securely to an account having medical history and/or medical records associated therewith, the systems and methods of the present invention further provide the ability to access the user's/patient's health history and/or information when the patient is granted access to the telehealth network and provides inputs via the mobile communications device having the prepaid or pre-authorized SIM card or chip activated therein.

After the system admittance pre-requisites are fulfilled and the patient is verified but before the patient is allowed into the telehealth network, the patient may be placed in a pre-activation status or holding period. This time period is referred to as dormant because the system has confirmed that the patient will be allowed access to the telehealth network, but the user or patient has not been activated completely into the network. Finally, activation of patient access into the telehealth network is initiated by at least one automated activation trigger.

For example and not limitation, the at least one trigger for activation to the network is provided by the SIM card or chip activation within the mobile communications device. The at least one trigger may further include a ticket purchase, check-in time, departure or arrival time, or employment hiring date, whereas the at least one trigger for de-activation or termination of access include check-out time, departure or arrival time, or employment termination date. Together, the controlled access afforded by the at least one trigger provides automatic access to the telehealth network by the user for accessing the medical information.

Preferably, the system includes a server and a patient communications device. In another embodiment, the system also includes a measurement device for measuring and/or communicating biometric data in real-time or near real-time.

In preferred embodiments of the present invention, unique identifiers include at least two of the following components: first name; last name; date of birth; unique identification identifier, code, or number; phone number; email address. Furthermore, the unique identification identifier, code, or number may be selected from the group consisting essentially of: a member number or ID, a rewards or loyalty number or ID, a health plan ID, a company ID, a ticket number, a confirmation number, a member ID and date of membership activation combination, and combinations thereof.

Another embodiment includes short-term eligibility based upon activation and duration of the SIM card or chip under the prepaid or pre-authorized period of time or telecommunications network usage or access. Optionally, in addition to the automated trigger and access to the telehealth network from the prepaid or pre-authorized SIM card or chip, examples of information for remote authorized access into the systems and methods for providing telehealth network access.

The activation access is controlled by at least one automated trigger. For example and not limitation, the at least one trigger for activation to the network may include a ticket purchase, check-in time, departure or arrival time, or employment hiring date, whereas the at least one trigger for de-activation or termination of access include check-out time, departure or arrival time, or employment termination date. Together, the controlled access afforded by the at least one trigger provides automatic, intermittent access to the patient. Preferably, the trigger for activation is not merely the patient entering information or filling out a form. The trigger for activation or de-activation is preferably the occurrence of an event or passage of time. In one embodiment, the occurrence of a trigger is detected by a non-generic computing device such as a GPS device detecting an airplane leaving an airport or arriving at an airport or any other type of transportation (train, vehicle, car, truck, boat, helicopter, etc.) leaving a departure point or arriving at a destination, a bar code reader, a QR code reader, or any other code reader reading a ticket or code corresponding to check-in, check-out, departure, or arrival, a card reader determining a payment card has been read at the card reader via NFC, RFID, magnetic card swipe, or any other card number entry which coincides with lodging check-in or check-out.

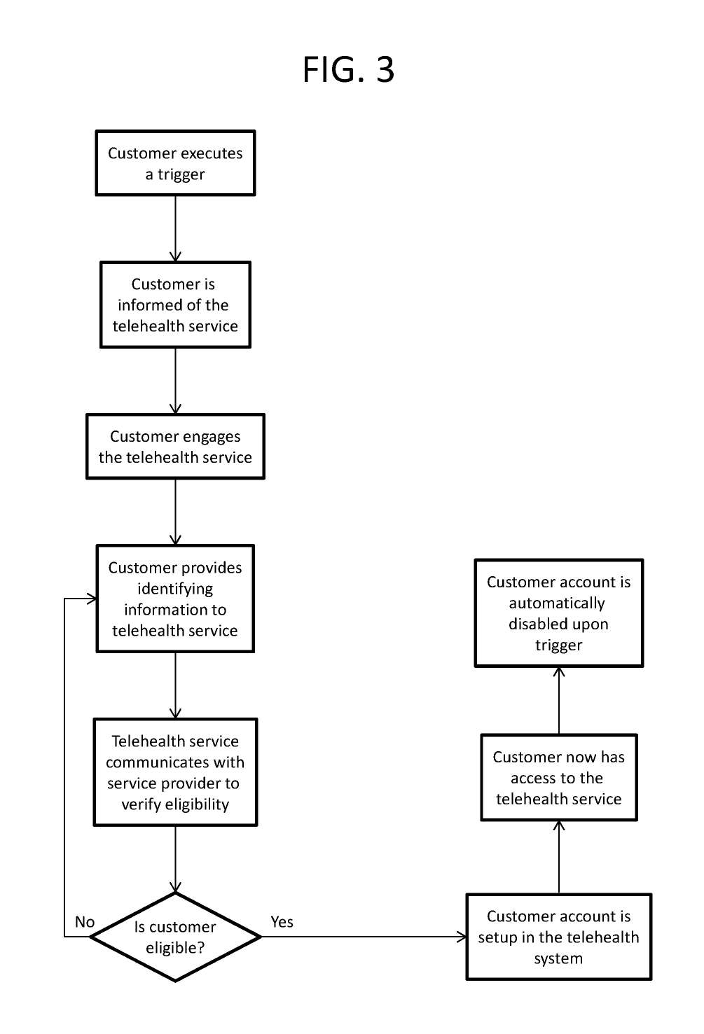

Another embodiment includes short-term eligibility; with example method steps displayed in FIG. 3. Here, a customer executes a trigger, such as purchasing an airline ticket, hotel reservation check-in, etc. The customer is informed of the telehealth service, such as by being included with reservation information, SMS or E-Mail notification, etc. The customer engages the telehealth service through the website, mobile app, or telephone. The customer provides identifying information to the telehealth service, such as name, ticket or reservation number, a mobile phone number, e-mail address, etc., to verify his or her eligibility for service. The telehealth service communicates with service provider to verify the customer's eligibility. This occurs through a secure web service. This process will be unique for every service provider. If the customer is eligible, a customer account is setup in the telehealth system. The customer now has access to the telehealth service. The customer account is automatically disabled upon receiving a termination trigger.

A benefit to the automated SIM card or chip access is that it provides instant activation of the telehealth network access upon activation of the SIM card or chip after insertion and connection within the mobile communications device. Alternatively or additionally, an e-mail or SMS is received automatically by the system following SIM card or chip activation, so that the user's mobile number, email address, etc. is used as the identifier as opposed to names or reservations numbers, making transaction between parties easier and more efficient, and risk of sharing potentially sensitive information, such as ticket numbers, reservations, etc., would be significantly reduced.

Additional eligibility dates beyond the active life of the SIM card or chip may be provided via additional subscription and/or activation by another trigger event, e.g., a check-in/check-out date range, travel date(s), etc. If the user is within the eligible window, the user's account would automatically set up and/or activate, and he or she would have access to the telehealth service for the dates provided from the service provider. The account would automatically close when the eligibility date(s) lapses or subscription expires (beyond the SIM card or chip active life). A mobile app, website, or network-based service for each service provider within the telehealth network (either direct or extended based upon the SIM card or chip or other subscription service or contract) may be provided beyond the medical information or medical briefs output based upon user inputs, depending upon local/geographical/country or governing entity requirements for telehealth advising or provision of medical services, diagnostics, testing, referrals, and/or treatments.

In one embodiment of the present invention, automatic access to a telehealth network for a user who is traveling is provided after the purchase and activation of a SIM card or chip for use with a mobile communications device. Similarly, the user is automatically released or discharged from the telehealth network by a de-activation trigger, wherein the de-activation trigger is the expiration of the SIM card or chip activity, e.g., time-based expiration (one month, one year, etc.), usage-based expiration (predetermined telecommunications minutes or predetermined monetary value or equivalent), or other contract-based expiration, such as by way of example and not limitation, subscription term. Geolocation services associated with the SIM card or chip and mobile communications device may also be used for automated activation and/or de-activation of the SIM card or chip, i.e., physical relocation of the mobile communications device with SIM card or chip installed therein, wherein the geolocation of the device is automatically determined or detected to be within (or outside) a geographic boundary or a geofence or a telecommunications service area.

The patient can learn of the system administrator based upon the agreement between service provider and system administrator. By way of example and not limitation, system administrator information could be provided by the service provider to the patient upon ticket purchase, check-in, etc., or the system administrator could be notified of a pre-determined trigger and automatically send the patient information via email or mobile messaging. A separate mobile app, website, etc. for each service provider can make the process easier on the patient, since each service provider may request different pieces of information for eligibility verification. By way of example and not limitation, when a patient looks for the mobile app he or she may see two apps—one called “System Administrator” and one called “System Administrator—Hilton Guests”. The “Hilton Guests” app applies to those patients staying at a Hilton hotel. This method would enable some co-branding of the app if the service provider desired this. The downside to separated apps would be the added confusion. Regardless, the standalone “System Administrator” app will be able to support typical customers and partnering service providers. When the patient accesses the System Administrator through one of the above methods, he or she will be asked for the unique identifiers that have been worked out with the service provider for eligibility to be verified.

Alternatively or additionally, as an airline frequent flyer or other membership or customer affiliation with an airline or travel agency or travel services entity, when the user purchases a ticket, the system automatically provides eligibility to the patient to join the telehealth network; the patient has fulfilled a pre-requisite for system admittance, namely membership. At this point, the user/traveler is placed in a dormant or holding phase preceding access into the telehealth network. Upon check-in for the initial flight's departure, the patient is provided access by the system into the telehealth network. In another embodiment, an established time period before check-in would act as the at least one trigger for activation. In an alternative embodiment, flight departure time would be the at least one trigger for activation. The user is released or discharged from the network by at least one trigger for de-activation, such as check-in for or departure of return flight or a predetermined time following confirmation of final air travel landing and deplaning at the final destination.

In another embodiment, as a hotel club member, when the user reserves a hotel room, the system automatically provides eligibility to the user to join the telehealth network. At this point, the user is placed in a dormant phase preceding full access by the system into the telehealth network. Upon hotel check-in—the at least one trigger for activation, the user is automatically provided access by the system into the telehealth network. In another embodiment, when the user enters within the range of the hotel's wireless internet signal for the first time following an initial trigger such as, by way of example and not limitation, the user accessing WellVia website or mobile App for confirming registration with the system, the user is automatically received into the telehealth network. Preferably, the mobile communication device used by the user for verification communicates with the hotel's wireless internet signal to connect to the telehealth network. The user is automatically released or discharged from the telehealth network by a de-activation trigger, such as hotel check-out.

In an alternative embodiment, a patient may purchase access to the system using membership rewards or airline miles, for example. In yet another embodiment, a patient may purchase access into the system with personal money, and following patient verification, the patient is granted access into the telehealth network. De-activation of network access would be triggered by a form of missed payment or the patient's permission to withdraw, for example.

Based upon the geolocation and governing authority or laws regarding medical advice or treatment services from a health care practitioner, the medical information will be limited to non-diagnostic information or it may be supplemented with electronic medical records and/or medical diagnostic information or medical advice or referral to medical treatment facilities or practitioners. Preferably, the SIM card or chip provides account information associated with the telecommunications network access provided.

In a preferred embodiment, the system delivers medical information inside the telehealth network that is not a medical diagnosis, but a matching of symptoms input received by the system against medical information stored within a database associated with the system. In one embodiment, the medical information stored within the database associated with the system includes medical information for a similar demographic (ex: based on age, gender, race, weight, height, lifestyle choices such as smoker/non-smoker, fitness, sports participation such as miming, skiing, boxing, football, etc.), medical information for family members, and/or medical information for the user.

Preferably, a physician can access the network to obtain raw and/or analyzed patient health records, as described in U.S. Pat. No. 7,970,633, issued Jun. 28, 2011 to Bulat, which is hereby incorporated by reference in its entirety. The physician and patient may communicate about the records or other medical and/or wellness information. This type of remote communication is especially convenient for a patient who may be traveling.

In addition to medical services, the telehealth network integrates and cross communicates between variable components of a patient's health, including, for example and not limitation, medical, wellness, and behavioral. The cross communication is possible via a cloud-based system, wherein data sharing and restriction follows HIPAA regulations. A patient's health variables are analyzed to provide an integrative view of the patient's health history, which then may be communicated to the patient via the patient's portable devices.

The telehealth network analysis may create a profile for the user and the associated SIM card or chip, based on the user inputs including health history or symptoms. The profile also reflects the user's patterns during the automatic activation period until automatic de-activation. Preferably, the user profile includes health history components, such as current medication and dosage, past medication and dosage, past therapy, current therapy, past injury, current injury, past surgeries, scheduled surgeries, past appointments, scheduled appointments, genetic predispositions to conditions, illness, tobacco usage, alcohol usage, drug usage, diet, allergies, fitness, and any activity or hobby which could influence health insurance information, and/or exercise and/or sports history, wherein details of these components include dates, frequencies, and dosages. Using these profile components, the network may forecast the user's patterns and/or projected measurements of the patient. When the user's actions and/or projected measurements do not correlate with the profile forecast, the system communicates to the user such an indication. Communication involves emails, texts, phone calls via the SIM card, and/or chip in the mobile, and other notifications to a communications device of the patient and/or a measurement device that provided the automatic activation and access to the telehealth network. The profile only includes information input by the user during the period of activation or coverage within the telehealth network, based upon the SIM card or chip activation period. In an alternative embodiment, communication involves emails, texts, phone calls, and/or other notifications to a health care provider, an emergency contact, a family member, and/or a caretaker of the patient. In yet another embodiment, communication involves phone calls to an ambulance, 911, or other emergency service.

The communication serves as a source of health accountability to improve compliance and adherence to ongoing medical, exercise, and/or dietary therapies. The network profile may identify risks and provide recommendations to the user to augment risk reduction. When risks have been reduced, the system may provide rewards and incentives to encourage healthy behavior patterns.

In another embodiment, the profile integrates previous lab metrics and physician appointment dates to provide pro-active follow-up. Messages will be sent to the patient for routine or non-routine physician appointment reminders.

In an alternative embodiment, the patient wears a device that tracks the patient's exercise and sleep habits. In another embodiment, the patient uses or wears a device which includes one or more biosensors operable to measure and monitor biological and/or physiological outputs. By way of example and not limitation, these devices with biosensors include measurement devices for measuring blood pressure, vision quality, pressure in the eyes, changes in pressure in the eyes, body temperature, temperatures of extremities, blood glucose, blood sugar, hydration, heart rate, sweat rate, sweat content, breathing rate, breathing volume, calories burned, number of whole and/or partial sleep cycles, length of sleep cycles, steps taken, medication taken from a container, medication taken in the human body (by way of example, by measuring the products of a reaction between the human body and the medication), an amount and duration of therapy from devices such as pain relieving devices which use infrared light therapy for pain relief, amounts of medication remaining such as the “puffs” left in an albuterol inhaler, detection of air quality, electrodermal response EDR, tremors for Parkinson's patients, speed of movement, posture, force of impact from physical sports, arrhythmia detection, epilepsy monitoring, stress monitoring, alpha wave monitoring, beta wave monitoring, pulse, blood oxygen saturation, lung function, standing time, exercise time, body mass index (BMI), cholesterol level, weight, etc. Preferably the measurement device is a non-generic computing device. Preferably, the measurements from these devices with biosensors are added to the profile of the patient. Preferably, the profile of the patient also includes past measurements from these devices and/or projected future measurements or ranges of measurements from these devices. If the measurement is not within a projected future range of measurements or equal to a projected future measurement, the server is operable to provide a target measurement and communicate that target measurement to the communications device and/or the measurement device of the patient. In one embodiment, the target measurement includes a target time frame. In another embodiment, the target measurement includes recommendations for achieving the target measurement. In yet another embodiment, one or more incentives are associated with achieving the target measurement.

The device communicates with the network profile to moderate forecast analysis. Similarly, the patient uses an app on his/her mobile communication device to input dietary habits, such as for example and not limitation, what, when, and where food was consumed. The device prompts the patient to enter the data, which increases adherence, and communicates this information with the network profile to moderate forecast analysis.

Security in the network including access restriction to authorized or authenticated users is provided by the SIM card or chip used in combination with the mobile communication device. Additionally, at least one personal identifier, i.e., personal identification is used, by way of example and not limitation, personal identification number (PIN) or code, personal biometric information including but not limited to fingerprint(s), retina scan, facial recognition, etc., and combinations thereof; the personal identification is preferably stored on the SIM card or chip and/or on other memory within the mobile communication device. Furthermore, a combination of the user registering with specific identifier(s) with the telehealth network via the telecommunications network, and the validation or authentication thereof with the service provider (e.g., SIM card or chip validation with the telecommunications service provider, travel service provider, airline, hotel, etc.). Upon a successful validation, the telehealth network system automatically creates and/or enables access for the user by providing an account specific to that user that is associated with the SIM card or chip activation and corresponding period or term. Physical and technical security is multifaceted. The telehealth network system provides additional security measures as well as strategic and technical partnerships with the service providers (datacenters, telecommunication providers, etc.) to require and enforce compliance with some or all but not limited to HIPAA/HITECH, ISO/IEC 27001/27002, PCI, SOC1, SOC2, SSL, functional equivalents, and combinations thereof provided by governing authorities overseeing the geographic location of the mobile communications device and SIM card or chip following activation. Preferably, data is encrypted at rest, i.e., if an unauthorized person were to remove data from the system they would not be able to access the information.

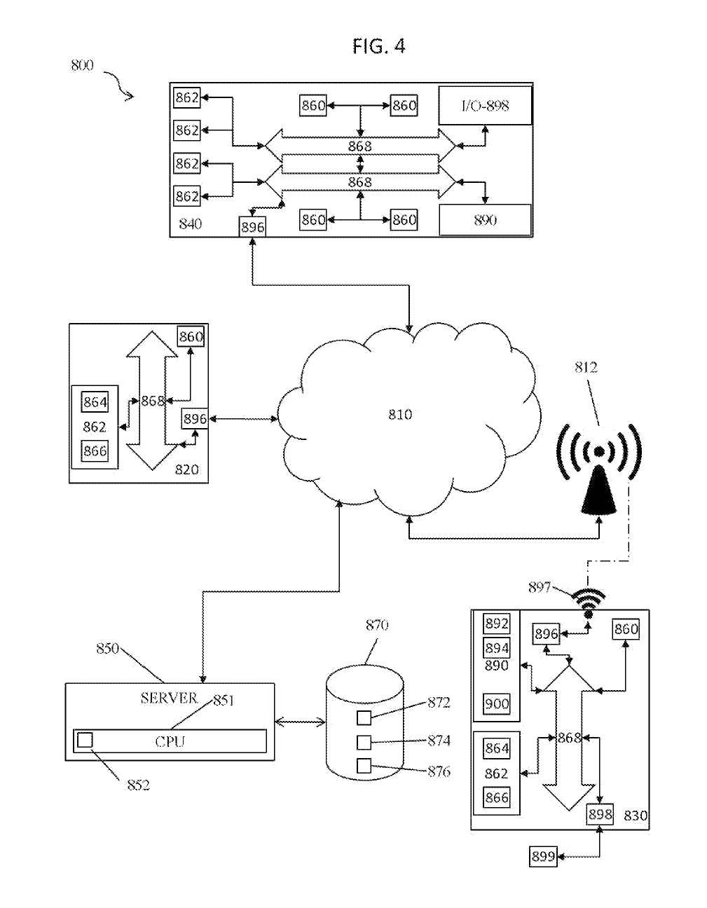

FIG. 4 is a schematic diagram of an embodiment of the invention illustrating a computer system, generally described as 800, having a network 810, a plurality of computing devices 820, 830, 840, a server 850 and a database 870.

The server 850 is constructed, configured and coupled to enable communication over a telecommunications network 810 with at least one computing device, including the mobile communications device having the SIM card or chip installed therein and activated automatically 820, 830, 840. The server 850 includes a processing unit 851 with an operating system 852. The operating system 852 enables the server 850 to communicate through network 810 with the remote, distributed user devices. Database 870 may house an operating system 872, memory 874, and programs 876.

In one embodiment of the invention, the system 800 includes a cloud-based network 810 for distributed communication via a wireless communication antenna 812 and processing by a plurality of mobile communication computing devices 830. In another embodiment of the invention, the system 800 is a virtualized computing system capable of executing any or all aspects of software and/or application components presented herein on the computing devices 820, 830, 840. In certain aspects, the computer system 800 may be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of digital computers 820, 840, 850 and mobile devices 830, such as a server, blade server, mainframe, mobile phone, a personal digital assistant (PDA), a smart phone, a desktop computer, a netbook computer, a tablet computer, a workstation, a laptop, and other similar computing devices. The components shown here, their connections and relationships, and their functions, are meant to be exemplary only, and are not meant to limit implementations of the invention described and/or claimed in this document.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 may additionally include components such as a storage device 890 for storing the operating system 892 and one or more application programs 894, a network interface unit 896, and/or an input/output controller 898. Each of the components may be coupled to each other through at least one bus 868. The input/output controller 898 may receive and process input from, or provide output to, a number of other devices 899, including, but not limited to, alphanumeric input devices, mice, electronic styluses, display units, touch screens, signal generation devices (e.g., speakers) or printers.

By way of example, and not limitation, the processor 860 may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 4, multiple processors 860 and/or multiple buses 868 may be used, as appropriate, along with multiple memories 862 of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function.

According to various embodiments, the computer system 800 may operate in a networked environment using logical connections to local and/or remote computing devices 820, 830, 840, 850 through a network 810. A computing device 830 may connect to a network 810 through a network interface unit 896 connected to the bus 868. Computing devices may communicate communication media through wired networks, direct-wired connections or wirelessly such as acoustic, RF or infrared through an antenna 897 in communication with the network antenna 812 and the network interface unit 896, which may include digital signal processing circuitry when necessary. The network interface unit 896 may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications or other data embodying any one or more of the methodologies or functions described herein. The computer readable medium may include the memory 862, the processor 860, and/or the storage media 890 and may be a single medium or multiple media (e.g., a centralized or distributed computer system) that store the one or more sets of instructions 900. Non-transitory computer readable media includes all computer readable media, with the sole exception being a transitory, propagating signal per se. The instructions 900 may further be transmitted or received over the network 810 via the network interface unit 896 as communication media, which may include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory or other solid state memory technology, disks or discs (e.g., digital versatile disks (DVD), HD-DVD, BLU-RAY, compact disc (CD), CD-ROM, floppy disc) or other optical storage, magnetic cassettes, magnetic tape, magnetic disk storage or other magnetic storage devices, or any other medium that can be used to store the computer readable instructions and which can be accessed by the computer system 800.

It is also contemplated that the computer system 800 may not include all of the components shown in FIG. 4, may include other components that are not explicitly shown in FIG. 4, or may utilize an architecture completely different than that shown in FIG. 4. The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

By way of definition and description supporting the claimed subject matter, preferably, the present invention includes communication methodologies for transmitting data, data packets, messages or messaging via a communication layer. Wireless communications over a network are preferred. Correspondingly, and consistent with the communication methodologies for transmitting data or messaging according to the present invention, as used throughout this specification, figures and claims, wireless communication is provided by any reasonable protocol or approach, by way of example and not limitation, Bluetooth, Wi-Fi, cellular, zigbee, near field communication, and the like; the term “ZigBee” refers to any wireless communication protocol adopted by the Institute of Electronics & Electrical Engineers (IEEE) according to standard 802.15.4 or any successor standard(s), the term “Wi-Fi” refers to any communication protocol adopted by the IEEE under standard 802.11 or any successor standard(s), the term “WiMax” refers to any communication protocol adopted by the IEEE under standard 802.16 or any successor standard(s), and the term “Bluetooth” refers to any short-range communication protocol implementing IEEE standard 802.15.1 or any successor standard(s). Additionally or alternatively to WiMax, other communications protocols may be used, including but not limited to a “1G” wireless protocol such as analog wireless transmission, first generation standards based (IEEE, ITU or other recognized world communications standard), a “2G” standards based protocol such as “EDGE or CDMA 2000 also known as 1×RTT”, a 3G based standard such as “High Speed Packet Access (HSPA) or Evolution for Data Only (EVDO), any accepted 4G standard such as “IEEE, ITU standards that include WiMax, Long Term Evolution “LTE” and its derivative standards, any Ethernet solution wireless or wired, or any proprietary wireless or power line carrier standards that communicate to a client device or any controllable device that sends and receives an IP based message. The term “High Speed Packet Data Access (HSPA)” refers to any communication protocol adopted by the International Telecommunication Union (ITU) or another mobile telecommunications standards body referring to the evolution of the Global System for Mobile Communications (GSM) standard beyond its third generation Universal Mobile Telecommunications System (UMTS) protocols. The term “Long Term Evolution (LTE)” refers to any communication protocol adopted by the ITU or another mobile telecommunications standards body referring to the evolution of GSM-based networks to voice, video and data standards anticipated to be replacement protocols for HSPA. The term “Code Division Multiple Access (CDMA) Evolution Date-Optimized (EVDO) Revision A (CDMA EVDO Rev. A)” refers to the communication protocol adopted by the ITU under standard number TIA-856 Rev. A.

It will be appreciated that embodiments of the invention described herein may be comprised of one or more conventional processors and unique stored program instructions that control the one or more processors to implement, in conjunction with certain non-processor circuits, some, most, or all of the functions for the systems and methods as described herein. The non-processor circuits may include, but are not limited to, radio receivers, radio transmitters, antennas, modems, signal drivers, clock circuits, power source circuits, relays, current sensors, and user input devices. As such, these functions may be interpreted as steps of a method to distribute information and control signals between devices. Alternatively, some or all functions could be implemented by a state machine that has no stored program instructions, or in one or more application specific integrated circuits (ASICs), in which each function or some combinations of functions are implemented as custom logic. Of course, a combination of the two approaches could be used. Thus, methods and means for these functions have been described herein. Further, it is expected that one of ordinary skill in the art, notwithstanding possibly significant effort and many design choices motivated by, for example, available time, current technology, and economic considerations, when guided by the concepts and principles disclosed herein, will be readily capable of generating such software instructions, programs and integrated circuits (ICs), and appropriately arranging and functionally integrating such non-processor circuits, without undue experimentation.

The above-mentioned examples are provided to serve the purpose of clarifying the aspects of the invention and it will be apparent to one skilled in the art that they do not serve to limit the scope of the invention. By way of example and not limitation, membership may include status in an alumni association, corporation or affiliation with at least one company or organization, professional association, religious organization or entity, etc. All modifications and improvements have been deleted herein for the sake of conciseness and readability but are properly within the scope of the present invention.

1. A telehealth system comprising:
at least one remote server with health care information;
at least one computer device with a SIM card or a chip;
wherein the at least one server and at least one computer device are in network communication; and
wherein the at least one remote server is further operable to create a profile for a patient wherein activation of access to a telehealth network is automatically triggered by activation of the SIM card or the chip.

2. The system of claim 1, wherein deactivation of the SIM card or the chip triggers deactivation of access to the telehealth network.

3. A method for providing telehealth services, the steps comprising:
providing at least one remote server with health care information and at least one computing device with a SIM card or a chip;
the at least one server and at least one computer device in network communication;
receiving an automated activation of access to a telehealth network from the at least one computer device, wherein the activation of access to the telehealth network is automatically triggered by activation of the SIM card or the chip; and
providing telehealth services to a user of the at least one computing device.

4. The method of claim 3, further comprising deactivating access to the telehealth network upon deactivation of the SIM card or the chip.

5-12. (canceled)

13. A system for providing automated access to a telehealth network comprising:
a communications device with a SIM card or a chip; and
a server administered by a telehealth network,
wherein the server is operable to enroll a patient into the telehealth network upon receipt of enrollment information from the communications device;
wherein the server is further operable to create a profile for the patient, wherein the profile includes the enrollment information;
wherein the server is further operable activate
access to the telehealth network for the patient upon the server receiving notification or detecting an activation trigger event, the activation trigger event including activation of the SIM card of the chip; and
wherein the server is further operable to de-activate access to the telehealth network for the patient upon the server receiving notification or detecting a deactivation trigger event.

14. (canceled)

15. The system of claim 13, wherein the deactivation trigger event includes an arrival time for transportation, a departure time for transportation, a check-in time for transportation, a check-out time for lodging, and/or an employment termination date.

16. The system of claim 13, wherein the profile for the patient includes health history components, wherein the health history components include current medication and dosage, past medication and dosage, past therapy, current therapy, past injury, current injury, past surgeries, scheduled surgeries, past appointments, scheduled appointments, genetic predispositions, illness, diet, allergies, fitness, health insurance information, and/or exercise history.

17. The system of claim 13, wherein the server is further operable to forecast future measurements and/or actions of the patient using the profile for the patient.

18. The system of claim 13 further comprising a measurement device, wherein the measurement device is operable to create at least one measurement by measuring heart rate, calories burned, body temperature, and/or blood glucose of the patient;
wherein the measurement device is further operable to transmit the at least one measurement to the server of the telehealth network; and wherein the server of the telehealth network is operable to add the at least one measurement to the profile of the patient.

19. The system of claim 18, wherein, upon receipt of the at least one measurement, the server of the telehealth network is operable to determine if the at least one measurement is within a range of forecasted measurements and/or is equal to at least one forecasted measurement;
wherein the range of forecasted measurements and/or the at least one forecasted measurement is created based on at least one past measurement, and if the at least one measurement is not within the range of forecasted measurements and/or is not equal to the at least one forecasted measurement, the server of the telehealth network is operable to send a notification to the communications device and/or the measurement device.

20. The system of claim 19, wherein the server is further operable to identify at least one target measurement based on the at least one measurement, the at least one past measurement, the range of forecasted measurements, and/or the at least one forecasted measurement;
wherein the measurement device is further operable to create at least one subsequent measurement by measuring heart rate, calories burned, body temperature, and/or blood glucose of the patient;
wherein the measurement device is further operable to transmit the at least one subsequent measurement to the server of the telehealth network;
wherein the server is further operable to compare the at least one subsequent measurement to the at least one target measurement; and
if the at least one subsequent measurement is equal to the at least one target measurement, is greater than the at least one target measurement if the at least one target measurement is a minimum measurement, or is less than the at least one target measurement if the at least one target measurement is a maximum measurement, the server is further operable to send a reward or a congratulatory message to the measurement device and/or to the communications device.

21-22. (canceled)

23. The method of claim 3, further comprising deactivating access to the telehealth network upon occurrence of a deactivation trigger event, wherein the deactivation trigger event is achieved by a GPS device measuring GPS coordinates of a vehicle or GPS coordinates of an aircraft, determining that the GPS coordinates of the vehicle or GPS coordinates of the aircraft are within a predetermined range of GPS coordinates, and communicating a deactivation message to the server.

24. (canceled)

25. The method of claim 3, wherein a deactivation trigger event for deactivating access to the telehealth network includes an arrival time for transportation, a departure time for transportation, a check-in time for transportation, a check-out time for lodging, and/or an employment termination date.

26. The method of claim 3, further comprising forecasting future measurements and/or actions of a patient using a profile for the patient.

27. The method of claim 3, further comprising:
a measurement device creating at least one measurement by measuring heart rate, calories burned, body temperature, and/or blood glucose of a patient;
the measurement device transmitting the at least one measurement to the at least one remote server; and
the at least one remote server adding the at least one measurement to a profile of the patient.

28. The method of claim 27, further comprising:
the at least one remote server determining if the at least one measurement is within a range of forecasted measurements and/or is equal to at least one forecasted measurement, wherein the range of forecasted measurements and/or the at least one forecasted measurement is created based on at least one past measurement; and
if the at least one measurement is not within the range of forecasted measurements and/or is not equal to the at least one forecasted measurement, the at least one remote server sending a notification to the at least one computing device and/or the measurement device.

29. The method of claim 28, further comprising:
the at least one remote server identifying at least one target measurement based on the at least one measurement, the at least one past measurement, the range of forecasted measurements, and/or the at least one forecasted measurement;
the measurement device creating at least one subsequent measurement by measuring heart rate, calories burned, body temperature, and/or blood glucose of the patient;
the measurement device transmitting the at least one subsequent measurement to the at least one remote server;
the at least one remote server comparing the at least one subsequent measurement to the at least one target measurement; and
if the at least one subsequent measurement is equal to the at least one target measurement, is greater than the at least one target measurement if the at least one target measurement is a minimum measurement, or is less than the at least one target measurement if the at least one target measurement is a maximum measurement, sending a reward or a congratulatory message to the measurement device and/or to the at least one computing device.
